(19) United States
(12) Patent Application Publication
(10) Pub. No.: US 2014/0155708 A1
(43) Pub. Date: Jun. 5, 2014

(54) SYSTEMS, METHODS AND RELATED APPARATUS FOR DETERMINING PHYSIOLOGICAL PARAMETERS

(71) Applicant: LIONSGATE TECHNOLOGIES, INC., Vancouver (CA)

(72) Inventors: Christian Leth Petersen, Burnaby (CA); John Mark Ansermino, Vancouver (CA); Guy Dumont, Vancouver (CA)

(73) Assignee: LIONSGATE TECHNOLOGIES, INC., Vancouver, BC (CA)

(21) Appl. No.: 14/234,986

(22) PCT Filed: May 13, 2013
(86) PCT No.: PCT/CA2013/050367

§ 371 Date: Jan. 24, 2014

Related U.S. Application Data
(60) Provisional application No. 61/646,841, filed on May 14, 2012.

(51) Int. Cl. A61B 5/00; A61B 5/11; A61B 5/0205; A61B 5/01; A61B 5/022

(52) U.S. Cl. CPC A61B 5/6898; A61B 5/01; A61B 5/022; A61B 5/02055; A61B 5/1107; A61B 5/7278
USPC 600/301; 600/300; 600/549; 600/485; 600/490; 600/483; 600/484

(57) ABSTRACT

Methods, systems and related apparatus are provided for controlling an electronic device to operate an external sensor connectable to an audio interface of the electronic device by applying a first harmonic driving signal to a first contact and a second harmonic driving signal to a second contact of the audio interface for driving the external sensor, receiving a response signal at a third contact of the audio interface, adjusting at least one of the first and second harmonic driving signals, determining one or more physiological parameters based on characteristics of the first and second harmonic driving signals and the response signal, and outputting the determined one or more physiological parameters.

### FIELD

The present disclosure relates generally to determining physiological parameters of a patient. More particularly, the present disclosure relates to systems, methods and related apparatus for determining physiological parameters with sensors coupled to audio interfaces of electronic devices.

### BACKGROUND

Conventional thermometers, blood pressure measurement devices, spirometers, perineometers, ECGs, EEGs, and other devices for measuring physiological parameters are typically standalone units. Standalone electronic devices for measuring physiological parameters usually contain a power source, a microcontroller, local storage, and a custom display mechanism along with the basic circuit needed to perform the sensing. This makes for relatively complex systems, costly to manufacture, and with many points of potential failure. They are therefore limited in their functionality, difficult to upgrade, and/or relatively expensive.

It is, therefore, desirable to provide improved systems and methods for determining physiological parameters.

### SUMMARY

Some aspects provide methods, systems and related apparatus for controlling an electronic device to operate an external sensor connectable to an audio interface of the electronic device by applying a first harmonic driving signal to a first contact and a second harmonic driving signal to a second contact of the audio interface for driving the external sensor, receiving a response signal at a third contact of the audio interface, adjusting at least one of the first and second harmonic driving signals, determining one or more physiological parameters based on characteristics of the first and second harmonic driving signals and the response signal, and outputting the determined one or more physiological parameters.

One aspect provides a system comprising a driving signal generator for applying a first harmonic driving signal to a first contact and a second harmonic driving signal to a second contact of the audio interface for driving the external sensor and adjusting at least one of the first and second harmonic driving signals, a response signal detector for receiving a response signal at a third contact of the audio interface, a physiological parameter extractor for determining one or more physiological parameters based on characteristics of the first and second harmonic driving signals and the response signal, and an output for outputting the determined one or more physiological parameters.

Another aspect provides a thermometer for connecting to an audio interface of an electronic device, the thermometer comprising a jack plug having first and second contacts for receiving first and second harmonic driving signals from the electronic device and a third contact for returning a response signal to the electronic device, and a temperature sensor consisting essentially of a thermistor connected between the first and third contacts and a reference resistor connected between the second and third contacts.

Another aspect provides an adaptor for connecting to an audio interface of an electronic device, the adaptor comprising a jack plug having first and second contacts for receiving first and second harmonic driving signals from the electronic device and a third contact for returning a response signal to the electronic device, and a pressure sensor having a pressure sensitive area, the pressure sensor connected as a bridge across the first and second contact to provide the response signal to the third contact, between the first and third contacts and a reference resistor connected between the second and third contacts.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a flowchart illustrating a method for controlling an external sensor through an audio interface of an electronic device according to one embodiment.

FIG. 1A is a flowchart illustrating a method for controlling an external sensor through an audio interface of an electronic device according to another embodiment.

FIG. 1B schematically illustrates an electronic device connected to control an example thermometer according to one embodiment.

FIG. 10 schematically illustrates an electronic device connected to control an example sphygmomanometer according to one embodiment.

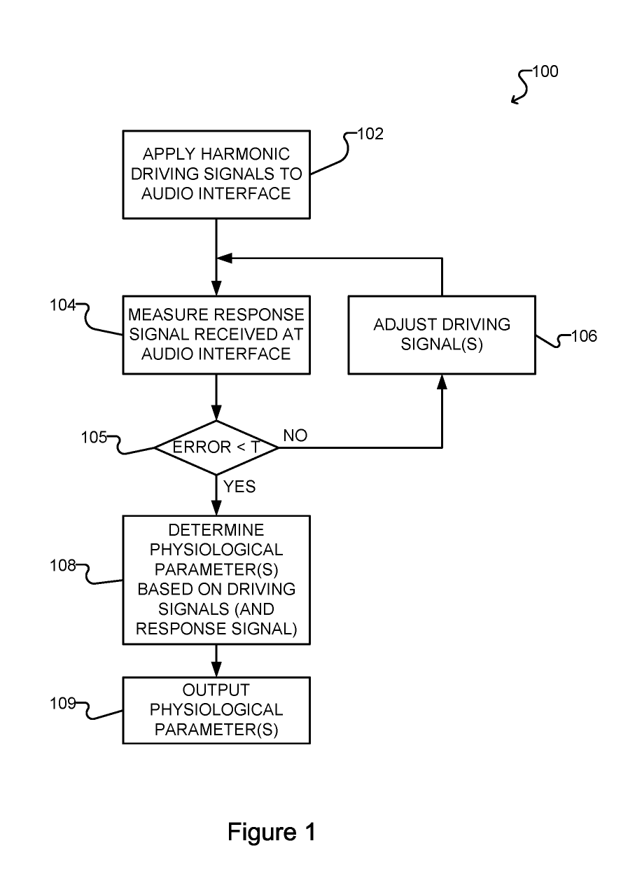

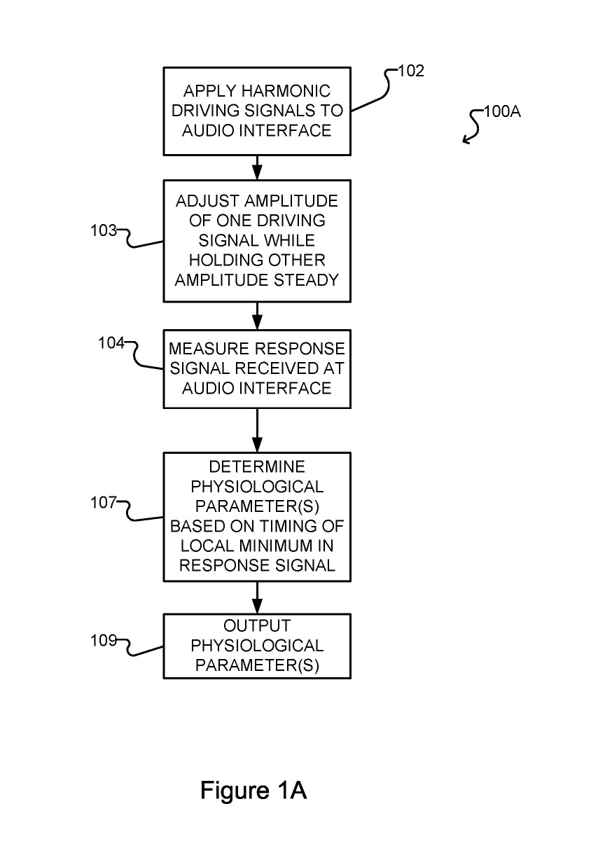

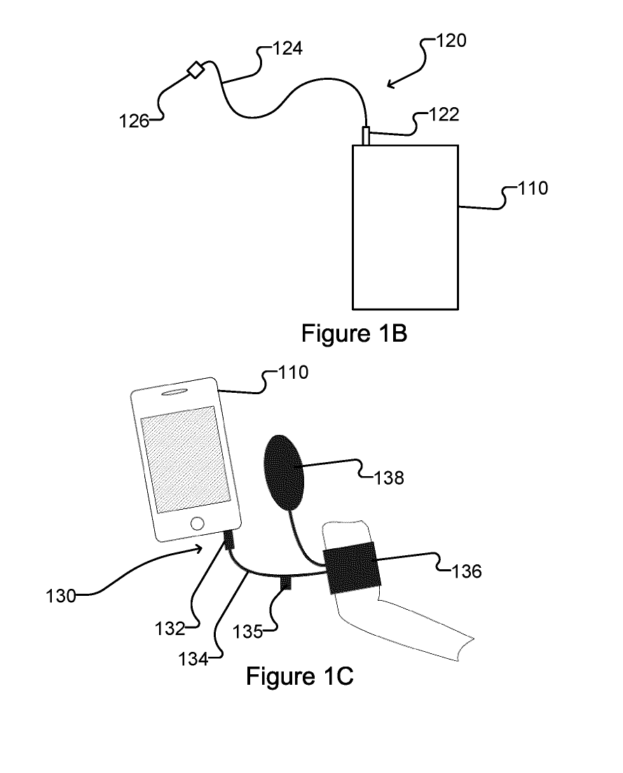

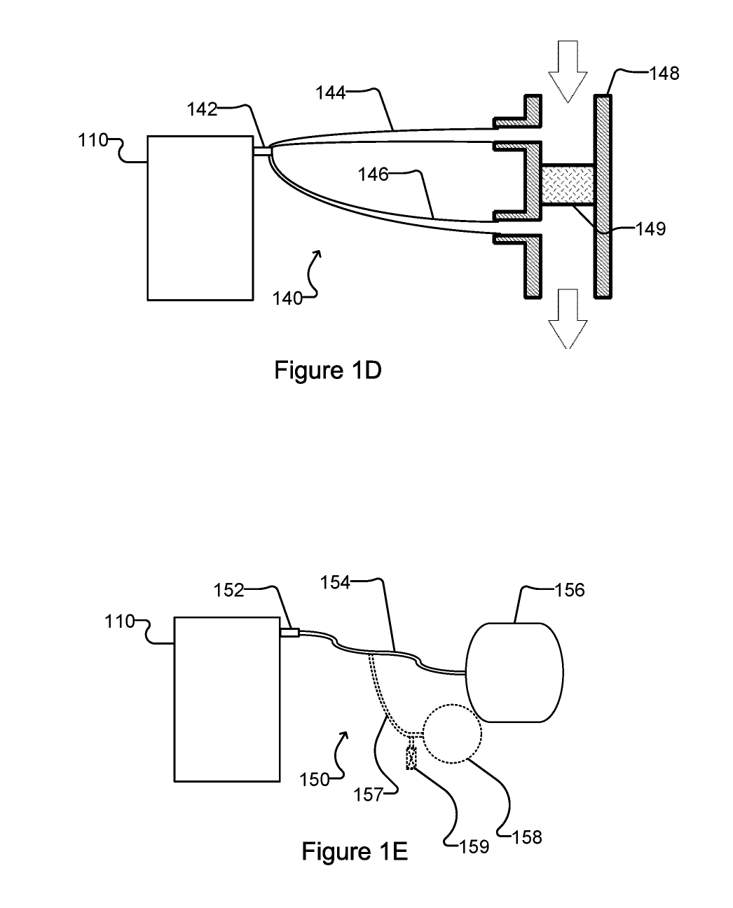

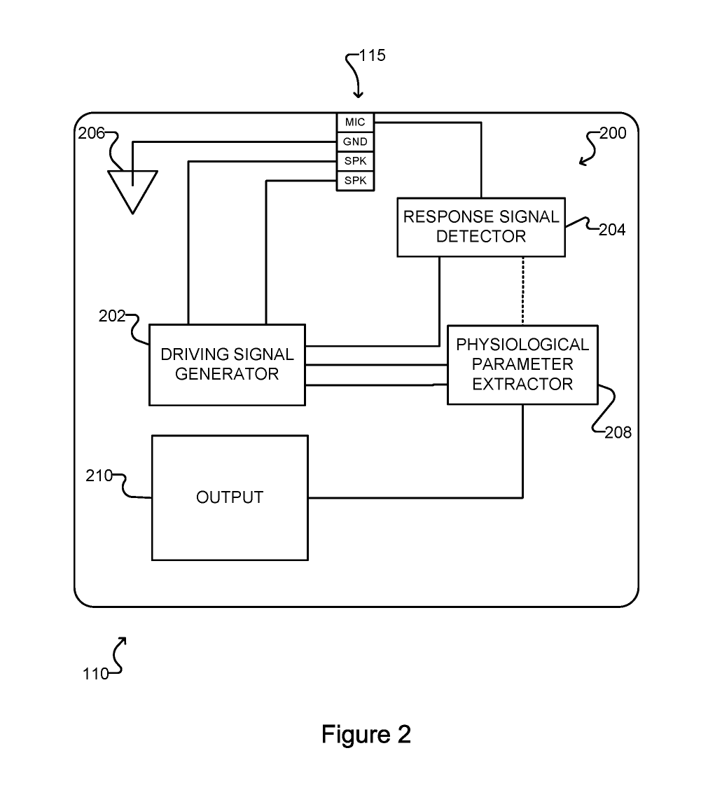

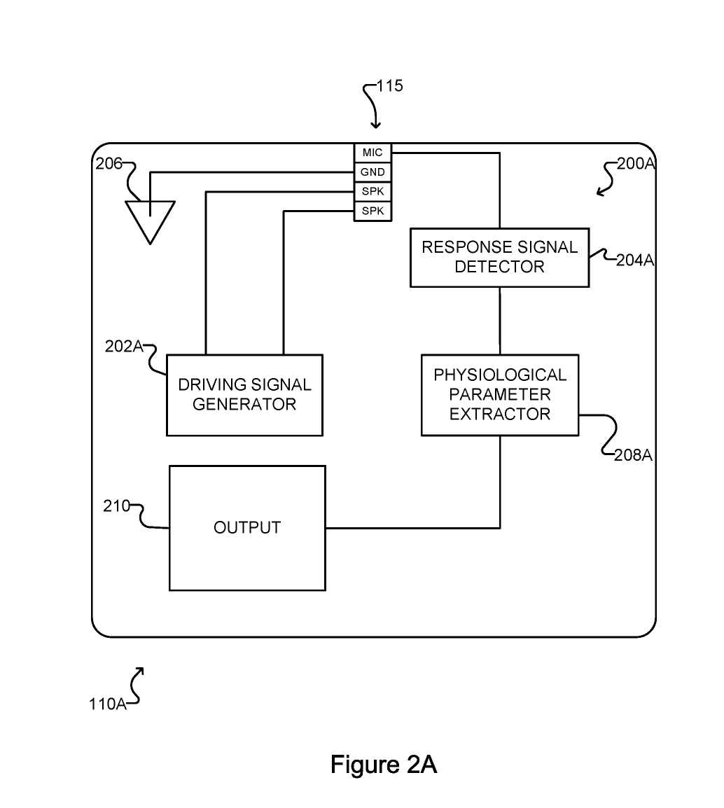

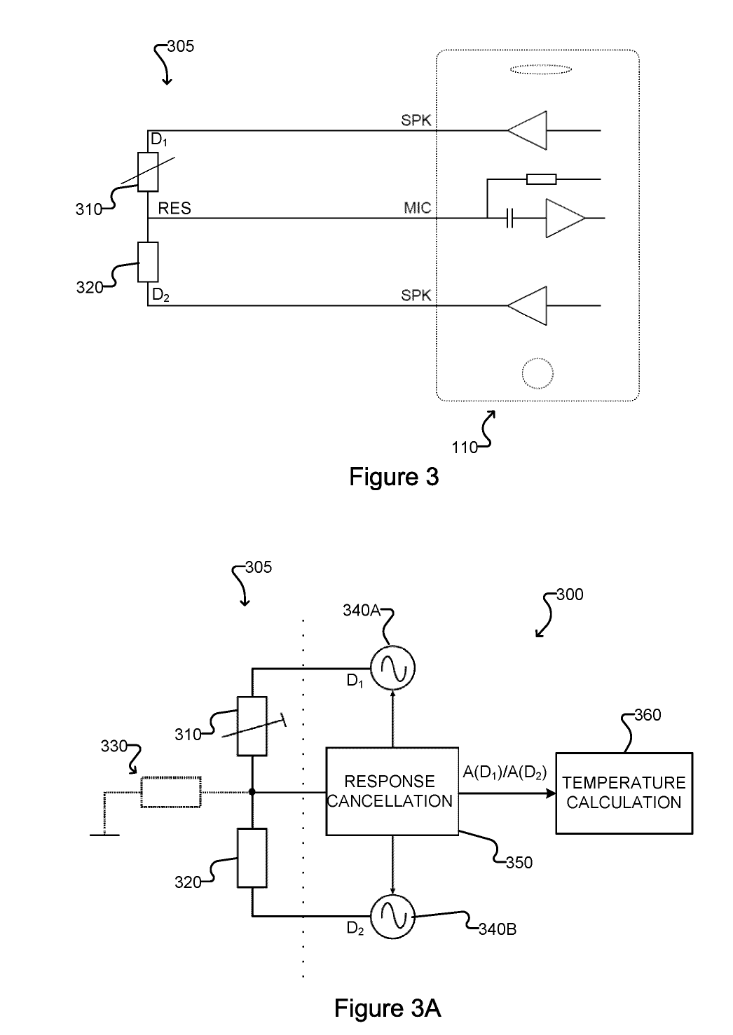

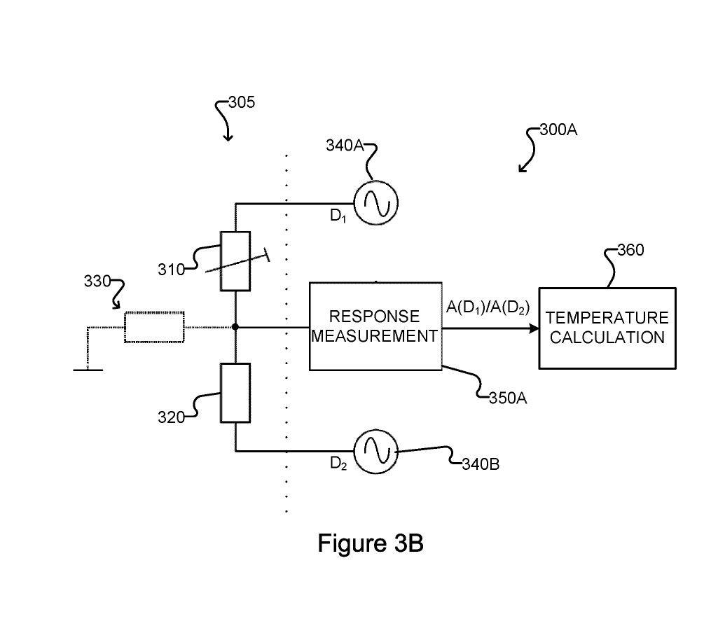

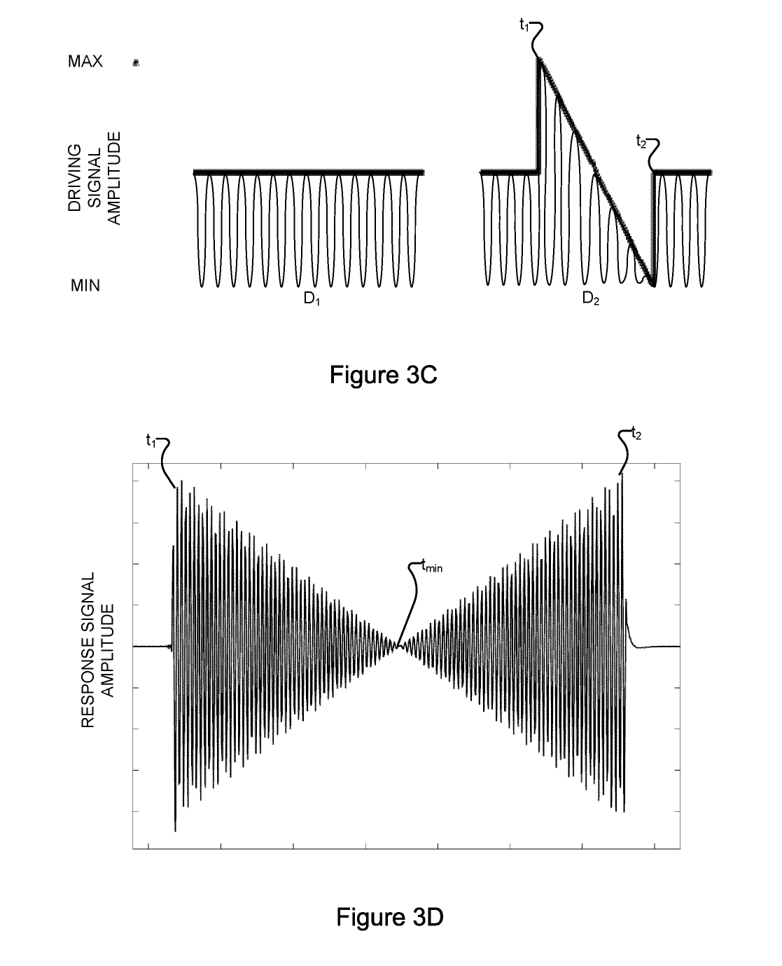

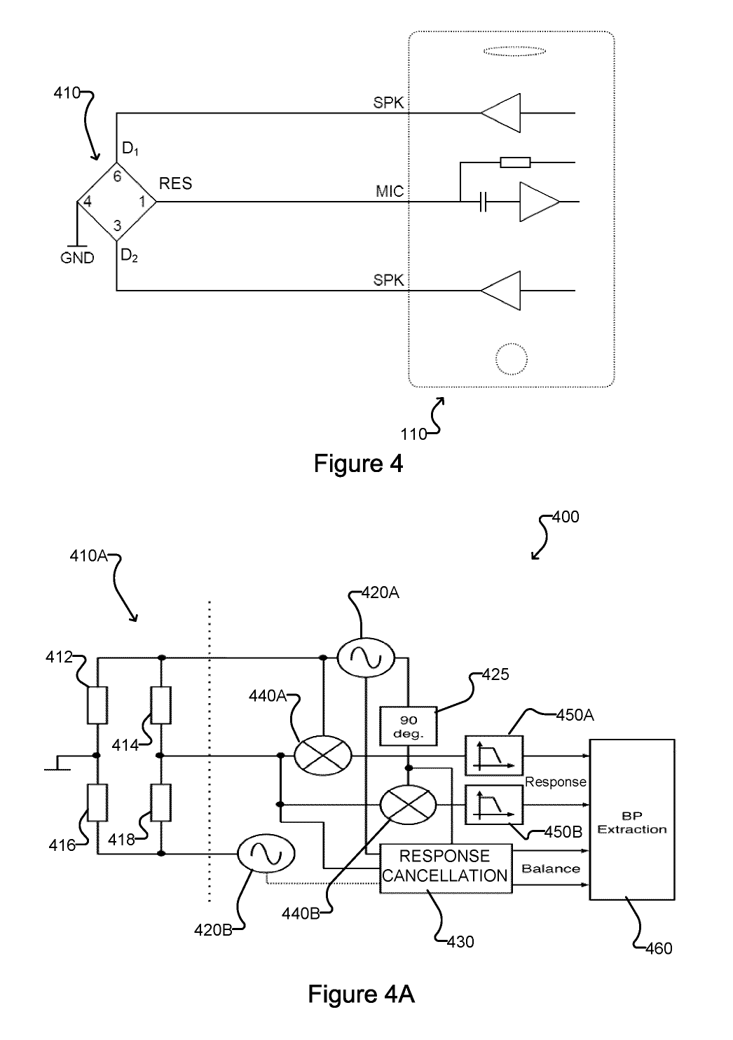

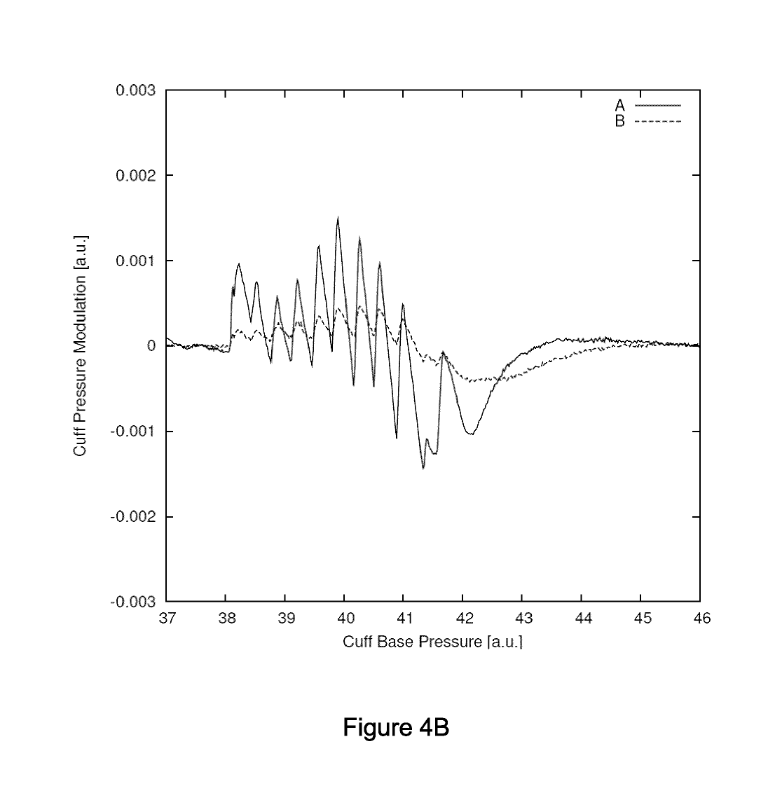

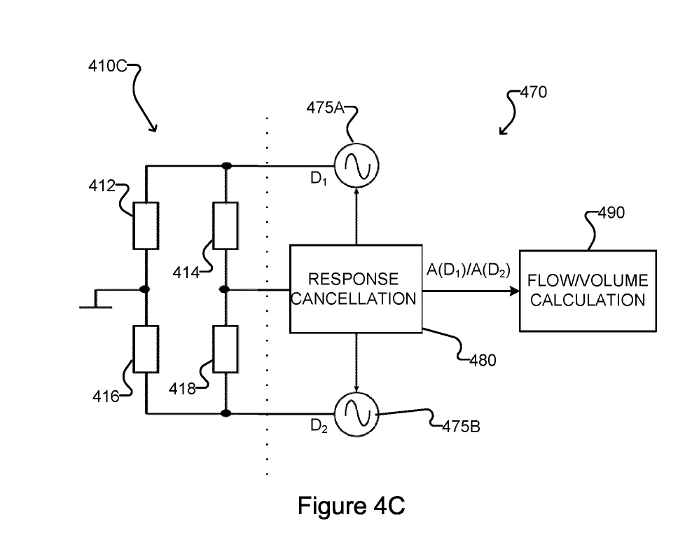

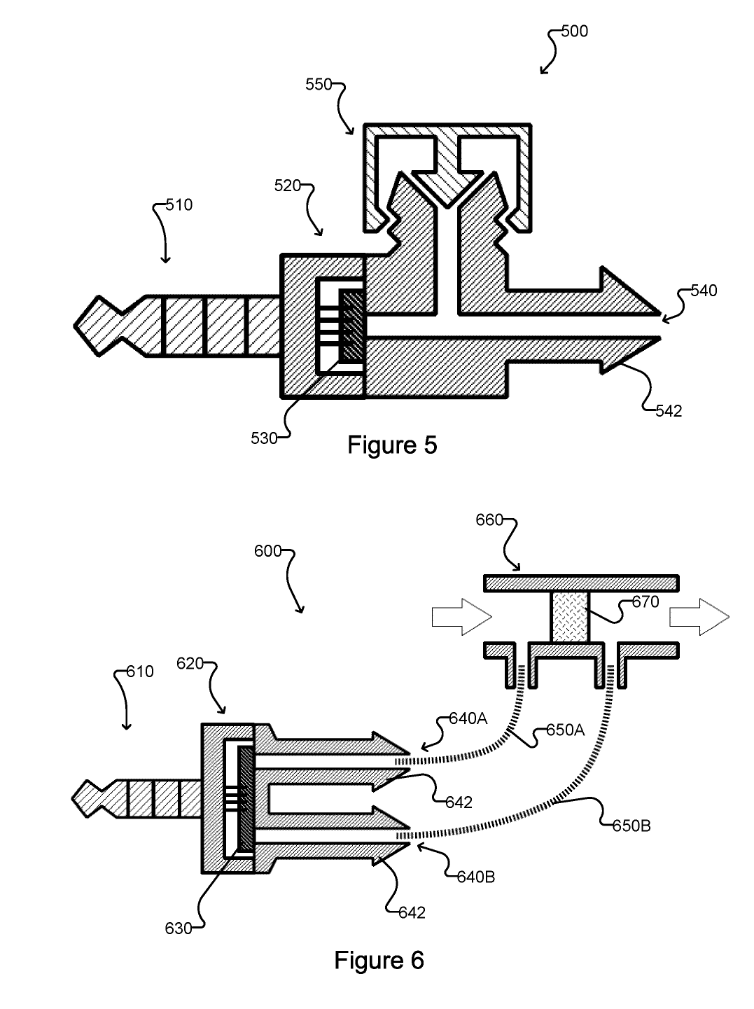

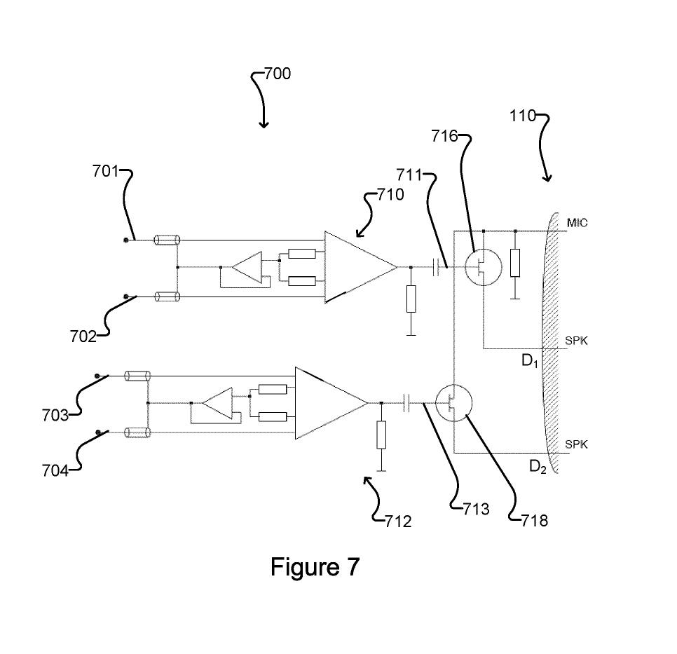

FIG. 1D schematically illustrates an electronic device connected to control an example spirometer according to one embodiment.

FIG. 1E schematically illustrates an electronic device connected to control an example perineometer according to one embodiment.

FIG. 2 schematically illustrates an example system for controlling an electronic device to operate an external sensor connectable to an audio interface of the electronic device according to one embodiment.

FIG. 2A schematically illustrates an example system for controlling an electronic device to operate an external sensor connectable to an audio interface of the electronic device according to another embodiment.

FIG. 3 schematically illustrates an example temperature sensor circuit according to one embodiment.

FIG. 3A schematically illustrates an example control system for operating the temperature sensor according to one embodiment.

FIG. 3B schematically illustrates an example control system for operating the temperature sensor according to one embodiment.

FIG. 3C schematically illustrates example driving signals of the control system of FIG. 3B.

FIG. 3D schematically illustrates an example response signal of the control system of FIG. 3B.

FIG. 4 schematically illustrates an example pressure sensor circuit for a sphygmomanometer or a spirometer according to one embodiment.

FIG. 4A schematically illustrates an example control system for operating a sphygmomanometer sensor according to one embodiment.

FIG. 4B is a graph showing example blood pressure output from a sphygmomanometer sensor operated through an audio interface according to one embodiment.

FIG. 4C schematically illustrates an example control system for operating a spirometer sensor according to one embodiment.

FIG. 5 shows an example sphygmomanometry audio interface adaptor according to one embodiment.

FIG. 6 shows an example spirometry audio interface adaptor according to one embodiment.

FIG. 7 schematically illustrates an example electrocardiogram (ECG) sensor circuit according to one embodiment.

### DETAILED DESCRIPTION

Generally, the present disclosure provides methods and systems for controlling external sensors through audio interfaces of electronic devices. Example embodiments are described herein with reference to hand held electronic devices such as smartphones or the like, but it is to be understood that the systems and methods described herein may be implemented in any type of electronic device with an audio interface and suitable signal generating and processing capabilities, including without limitation smartphones, feature phones, personal digital assistants, tablet computers, netbook computers, laptop computers, portable gaming systems, portable music players equipped with processors, desktop computers, or the like.

Performing medical sensor measurements with portable devices such as mobile phones is advantageous for purposes of recording and transmitting readings, providing power to the sensor, data storage, powerful processors for advanced analytics, ease of upgrade and addition of application features, as well as facilitating a better user interface. Enhanced user interaction is possible through the versatile input and output mechanisms of a mobile phone, allowing for example to input supporting patient information, to guide the patient for ensuring optimal sensor placement, and to provide a richer feedback with the results, including treatment options, graphing of historic data trends and other relevant context.

By providing sensors connected directly to a mobile phone or other electronic device, embodiments disclosed herein eliminate the need for many components of typical standalone devices for measuring vital signs or other physiological parameters (such as separate power sources, microcontrollers, custom displays, etc.), as their functions are inherent to the mobile phone itself. These embodiments reduce the complexity of the sensor design and lower the cost of sensor manufacture. Due to the relative ease of software upgrades, the application controlling the sensor can be incrementally improved and updated. Embodiments described herein are configured to take advantage of the near universality of the audio interface in modern electronic devices. Certain embodiments also leverage the relatively high level of standardization of audio jacks and jack plugs. Furthermore, the consumer grade audio codecs in many electronic devices use sophisticated signal converters to achieve resolutions of 16 bits (15 ppm) or more. This by far outperforms the converters found in most custom microcontroller implementations that typically have resolutions of 10 bits (1000 ppm). This hardware level improvement of almost 100 times in signal quality can be leveraged to significantly improve the accuracy of the sensor readings. In addition, standard audio input and output signal levels and impedance ranges are directly compatible with a wide range of sensor types. In particular certain embodiments use sensors comprising bridge circuits of passive components for measurements, such as is the case for pressure and temperature sensing as discussed below.

There is however a significant difference between the audio interface and typical sensor interfaces in that the audio signal channel is designed to operate strictly on AC signals, whereas conventional sensor interfaces rely on DC signals for sensor actuation and detection. While it is possible to use an audio signal line to transmit DC sensor signals by means of amplitude and/or frequency modulation of AC carrier signals that are encoded and decoded at each end of the audio channel, this would require the presence of additional converter electronics between the sensor and the phone audio connector, which would be detrimental to the purpose of using a phone or other electronic device for eliminating custom electronics, as well as reintroduce the need for a separate sensor power source.

The standard microphone connection of an audio interface such as a mobile phone headset jack outputs a small voltage, which is used to drive the electret microphones commonly found in headsets. This “microphone bias” is sufficient to drive a single transistor or single ultra-low power device, but not to power a typical full sensor interface or signal conversion circuit.

Certain embodiments fully leverage the advantages and capabilities of modern electronic devices by employing sensor control methods and systems that are inherently AC in nature, such that the sensor input and output can be connected directly to the audio channel without additional signal conversion or conditioning, and without the need for a power source other than the microphone bias. This requires a new approach to sensor actuation, signal detection and processing.

FIG. 1 shows an example method 100 for controlling an external sensor through an audio interface according to one embodiment. As described further below, method 100 may be executed by an electronic device to operate a variety of sensors, including without limitation thermometers, sphygmomanometers, perineometers, ECG and EEG monitors, and spirometers. In some embodiments, a sensor circuit is connected as an AC bridge across the contacts of an audio interface, and driving signals are applied to the arms of the bridge and adjusted to minimize a response signal detected from a central point of the bridge.

Method 100 comprises applying harmonic driving signals to a pair of contacts of the audio interface at block 102. At block 104 a response signal received at another contact of the audio interface is measured. At block 105 method 100 determines whether the response signal is within a predetermined threshold of zero. If not, method 100 proceeds to adjust either one or both of the driving signals at block 106 to reduce the response signal. As one skilled in the art will appreciate, blocks 104-106 constitute a feedback loop for minimizing the response signal, may be executed substantially simultaneously, and may be implemented in a variety of ways.

When the response signal is minimized, method 100 proceeds to block 108, where one or more physiological parameters are determined based on characteristics of the driving signals applied to minimize the response signal. In some embodiments, physiological parameters may be determined based on a ratio of the amplitudes of the applied driving signals. In some embodiments, method 100 may also comprise detecting small variations of the response signal, and determining physiological parameters based on variations of the response signal at block 108. At block 109 the physiological parameter(s) are output to the patient and/or other user(s). For example, outputting physiological parameters may comprise displaying the physiological parameters on a display of the electronic device, generating audible signals with built-in speakers of the electronic device, generating a haptic signal, storing the physiological parameters in memory of the electronic device, sending the physiological parameters to one or more other devices using any suitable communication protocols available at the electronic device, passing the physiological parameters to another application through a programmatic interface, or any other form of output.

FIG. 1A shows an example method 100A for controlling an external sensor through an audio interface according to another embodiment. Method 100A may be executed by an electronic device to operate a variety of sensors, including without limitation thermometers, sphygmomanometers, spirometers, EEG and ECG monitors, and perineometers. In some embodiments, such as shown in FIG. 3B for the case of a thermometer, a sensor circuit is connected as an AC bridge across the contacts of an audio interface, and driving signals are applied to the arms of the bridge and adjusted by applying a predetermined amplitude profile to determine when a response signal detected from a central point of the bridge is minimized.

Method 100A comprises applying harmonic driving signals to a pair of contacts of the audio interface at block 102. At block 103, the amplitude of one of the driving signals is adjusted according to a predetermined amplitude profile (e.g., a linear ramp as in the example of FIG. 3C discussed below, or other predictably varying amplitude), and the amplitude of the other driving signal is held constant (e.g., at about % of the maximum amplitude as in the example of FIG. 3C discussed below, or other substantially constant amplitude). At block 104 a response signal received at another contact of the audio interface is measured. As discussed further below, the time at which the response signal exhibits a local minimum is detected at block 104, and from the timing of this local minimum in the response signal, the ratio of amplitudes of driving signals which caused the local minimum may be determined. At block 107 one or more physiological parameters are determined based on the timing of the local minimum in the response signal. At block 109 the physiological parameter(s) are output to the patient and/or other user(s) as discussed above with reference to method 100.

FIGS. 1B-E illustrate example embodiments wherein an electronic device 110 is used to operate a variety of external sensors. The examples of FIGS. 1B-E are directed to thermometry, sphygmomanometry, spirometry, and perineometry applications but it is to be understood that other embodiments may be directed to other applications. For example, other types of sensors including, without limitation, electrocardiogram (ECG) and electroencephalogram (EEG) sensors may be connected to audio interfaces of electronic devices in other embodiments.

FIG. 1B shows an electronic device 110 connected to an example thermometer 120 according to one embodiment. Thermometer 120 comprises a connector 122, a cable 124 and a probe 126. Cable 124 may advantageously provide a user with flexibility in placement of probe 126, but is not required in all embodiments. For example, in some embodiments probe 126 may be directly attached to connector 122, may be incorporated into connector 122, or may be coupled to connector 122 by other means (e.g. coupled to a rigid or flexible member attached to connector 122). Probe 126 comprises temperature sensing circuit elements which may be controlled by device 110 to obtain temperature readings, as described further below.

FIG. 1C shows an electronic device 110 connected to an example sphygmomanometer 130 according to one embodiment. Sphygmomanometer 130 comprises a connector 132, a pressure tube 134, an inflatable cuff 136, and a pressure source 138. Pressure source 138 may be a hand-operated pump, as shown in the illustrated embodiment, or it may be a powered pump, or some other source of pressure. A leak valve 135 may be provided on tube 134. Alternatively, a leak valve may be incorporated into connector 132, as described further below, or elsewhere in the device, according to other embodiments. Cuff 136 may be wrapped around a patient's limb (e.g. arm) and pressure source 138 may be used to inflate cuff 136, and the pressure in cuff 136 is communicated through tube 134 to a pressure sensor in connector 132. The pressure sensor in connector 132 may be controlled by device 110 to measure variations in the pressure in cuff 136, which may be analyzed to extract a variety of physiological parameters, including systolic, mean and diastolic blood pressure, respiratory rate, and heart rate, as described further below. The pressure sensor may also be situated elsewhere in the device, according to other embodiments. For example, the pressure sensor may be located closer to or at the cuff 136, and connected to connector 132 by wiring.

FIG. 1D shows an electronic device 110 connected to an example spirometer 140 according to one embodiment. Spirometer 140 comprises a connector 142, a pair of pressure tubes 144 and 146, and a breathing tube 148 having a partial obstruction 149 therein. Tubes 144 and 146 communicate pressures from opposite sides of obstruction 149 to a differential pressure sensor in connector 142. Obstruction 149 is configured such that when a patient breathes through breathing tube 148 in the direction indicated by the arrows in FIG. 1D, the pressure in tube 144 will be greater than the pressure in tube 146. The differential pressure sensor in connector 142 may be controlled by device 110 to measure variations in the pressure across obstruction 149, which may be analyzed to extract a variety of physiological parameters, including lung capacity and air flow measurements, as described further below.

FIG. 1E shows an electronic device 110 connected to an example perineometer 150 according to one embodiment. Perineometer 150 comprises a connector 152, a pressure tube 154, and an inflatable member 156 which may be inserted into the vagina. Tube 154 communicates pressure from member 156 to a pressure sensor in connector 152. Perineometer 150 may also optionally comprise another pressure tube 157 connecting a manually operable air bulb 158 to pressure tube 154. The air bulb 158 may be used to inflate member 156, and a pressure release valve 159 may be provided on pressure tube 157 to prevent over-inflation. The pressure sensor in connector 152 may be controlled by device 110 to measure variations in the pressure applied to member 156, which may be analyzed to extract a variety of physiological parameters, including the strength of voluntary contractions of the pelvic floor muscles, as described further below. Alternatively, a mechanically compressible member may be provided in place of inflatable member 156. The mechanically compressible member may comprise a strain gauge-type pressure sensor such as, for example, one or more piezoelectric or piezoresistive elements that are coupled to the audio interface of device 110 through connector 152 and suitable wiring.

FIG. 2 shows an example system 200 according to one embodiment. System 200 may be implemented in an electronic device 110 to control an audio interface 115 to operate an external sensor and process the response signals (which, when used as feedback to modify the driving signals, are conventionally referred to as “error signals”, and are commonly sought to be minimized by the feedback process) from the sensor. In the illustrated embodiment, audio interface 115 comprises a TRRS (tip, ring, ring, sleeve) audio interface wherein the tip and first ring comprise speaker contacts SPK, the second ring comprises a ground contact GND, and the sleeve comprises a microphone contact MIC, but it is to be understood that different types of audio interfaces may be used. For example, some embodiments may use a TRRS audio interface with a different arrangement of contacts. Some embodiments may use a pair of TRS type interfaces (e.g., a speaker output interface and a microphone input interface). Some embodiments may use differently configured audio interfaces with a plurality of contacts for sending and receiving electrical signals. In the illustrated embodiment, system 200 comprises a driving signal generator 202 for applying harmonic driving signals to speaker contacts SPK, and a response signal detector 204 for detecting a response signal received at microphone contact MIC. An internal ground 206 is connected to ground contact GND.

Response signal detector 204 provides feedback to driving signal generator 202 for adjusting the driving signals to minimize the response signal. Driving signal generator 202 provides a physiological parameter extractor 208 with characteristics of the driving signals (e.g. phase and amplitude) that minimize the response signal. In some embodiments physiological parameter extractor 208 receives a balance signal indicating a ratio of amplitudes of the driving signals. In some embodiments physiological parameter extractor 208 may also receive one or more signals generated based on the response signal received by response signal detector 204, as indicated by the dotted line connecting response signal detector 204 and physiological parameter extractor 208. Physiological parameter extractor 208 determines one or more physiological parameters based on characteristics of the driving signals, and optionally based on the response signal, as described further below, and provides the determined physiological parameter(s) for output at output 210.

FIG. 2A shows an example system 200A according to another embodiment, which may also be implemented in an electronic device 110 to control an audio interface 115 to operate an external sensor and process the response signals from the sensor. Similar to system 200 of FIG. 2, system 200A comprises a driving signal generator 202A for applying harmonic driving signals to speaker contacts SPK, and a response signal detector 204A for detecting a response signal received at microphone contact MIC, and an internal ground 206 is connected to ground contact GND. System 200A differs from system 200 in that driving signal generator 202A does not receive any feedback, but instead adjusts one of the driving signals in a pre-determined pattern. The pre-determined pattern may be fixed or may be selected according to some detected condition or signal. For example, if a specific characteristic of the response signal, such as a minimum value, were detected, a pre-determined pattern that narrows the range of amplitudes to sweep through to values immediate to the region where the minimum value occurred might be selected.

Response signal detector 204A detects a local minimum in the amplitude of the response signal, and determines timing information thereof. The timing information of the local minimum in the response signal may be used by response signal detector 204A in conjunction with predetermined characteristics of the driving signals to determine the ratio of driving signals that caused the local minimum, and the ratio of driving signals may be provided to physiological parameter extractor 208A. Alternatively, the timing information may be provided directly to physiological parameter extractor in 208A. Physiological parameter extractor 208A determines one or more physiological parameters based on the signal from response signal detector 204A, as described further below, and provides the determined physiological parameter(s) for output at output 210. In other embodiments, other characteristics of the response signal including without limitation a maximum, an inflection point, or a specific non-zero value may be detected and provided to the physiological parameter extractor 208A.

### Example Thermometry Embodiments

A clinical thermometer is used to measure human body temperature. Body temperature reflects relative health, and a significantly elevated or lowered temperature can indicate illness. Conventional electronic clinical thermometers use a thermistor probe to measure temperature at a point that best approximates core body temperature, typically the mouth, armpit or rectum, and display the result with a resolution of typically 0.1 degrees. The accuracy of these devices is usually less, typically +/−0.2 degrees. Sufficiently accurate thermometers can be used to help predict female fertile periods by measuring basal body temperature (BBT). BBT is the lowest temperature reached while resting and is obtained in the morning after sleep. BBT typically peaks at least 0.4 degrees higher than normal over a 48-hour period when ovulation has occurred, and remains elevated until menstruation begins. A woman can use this method to predict ovulation by charting temperatures for several cycles to predict optimal likelihood of fertility. Accurate thermometers are also useful in a variety of other situations, as known in the art.

Certain embodiments provide low cost, robust and accurate thermometers which may be connected to audio interfaces of electronic devices. By eliminating all hardware for processing, storing, and displaying the measured temperature, and instead interfacing the thermistor directly to the audio port of a mobile phone or other portable electronic device, the complexity, cost and materials required for clinical grade thermometers can be significantly reduced.

FIG. 3 shows an example thermometer circuit 305 connected to two speaker contacts SPK and a microphone contact MIC of an electronic device 110. A temperature sensitive circuit element 310 is connected between a first of the speaker contacts and the microphone contact MIC, and a reference circuit element 320 is connected between a second of the speaker contacts SPK and the microphone contact MIC. In some embodiments, circuit 305 may include only a single thermistor as temperature sensitive circuit element 310 and a single reference resistor as reference circuit element 320. Such an arrangement would advantageously minimize the cost of circuit 305. However, it is to be understood that additional components may be included in other embodiments, such as for example, one or more additional resistors or thermistors connected in series or parallel. In some embodiments, circuit 305 may also comprise a ground connection 330, as shown in FIG. 3A.

FIG. 3A shows an example control system 300 for obtaining temperature measurements with thermometer circuit 305. Control system 300 comprises signal sources 340A and 340B which drive contacts of the audio interface (e.g. the left and right audio output ports) with harmonic driving signals D₁ and D₂ of a selected frequency, typically in the range from about 100 Hz to about 10000 Hz. A response cancellation block 350 adjusts the amplitude of one (or both) of these signals until the response signal RES recorded by the microphone input is at a minimum. Response cancellation block 350 may also adjust the phase of one (or both) of the driving signals, for example to compensate for impedances of elements 310 and 320. The ratio of the amplitudes of the driving signals A(D₁)/A(D₂), which is proportional to the ratio of the resistances of elements 310 and 320, is provided to a temperature calculation block 360 for determining the temperature.

With reference to an example embodiment comprising only two electrical components, they may be a thermistor having a variable resistance R and a reference resistor having a known resistance R_{ref}. The reference resistor may be chosen to have a resistance close to that of the thermistor at a desired temperature. The desired temperature may vary depending on the intended use of the thermometer. For example, in some embodiments the desired temperature may be about room temperature, about body temperature, or anywhere in between. The ratio of driving amplitudes A(D₁)/A(D₂) is now equivalent to the balance of resistances b (b=R/R_{ref}) and the temperature can be determined from:

$$T = {\{ \frac{1}{β} ln ( \frac{R}{{R}_{0}} + \frac{1}{{T}_{0}} ) \}}^{- 1}$$

where T is the temperature in Kelvin, R=bR_{ref} the thermistor resistance at the measured temperature, R₀ is the resistance of the thermistor at a reference Kelvin temperature T₀, and β is the thermal material constant of the thermistor. By nature of the null-detector arrangement the output and input gain of the mobile device does not enter the measurement.

Balancing of the driving signals in embodiments such as the example of FIG. 3A can be achieved by any suitable method, including those known in the art. An example embodiment uses a Widrow-Hoff least mean square approach as set out in the following pseudo-code:

```
Input : Time in seconds time, microphone input signal input
Output: Audio output signals output1, output2
ω: Audio signal angular frequency
converge: Bridge convergence factor
balance[2]: Audio bridge quadrature balance
begin
  | s = sin(ω × time)
  | c = cos(ω × time)
  | output1 = s
  | output2 = balance[0] × s + balance[1] × c
  | balance[0] = balance[0] − converge × input × s
  | balance[1] = balance[1] − converge × input × c
end
```

The bridge convergence factor is determined based on electrical noise and latency at the audio interface of the electronic device used to implement the control system. If for example the convergence factor is too large, the adjusted driving signal applied to one of the outputs of the audio interface may oscillate or diverge in an unstable manner.

In addition to ultra-low cost, an additional benefit of temperature sensors according to embodiments with control systems such as the example of FIG. 3A is that the audio interface has high resolution, and the precision of the temperature measurement is on the order of 0.01 degrees, significantly better than many conventional standalone thermometers. This makes thermometers according to such embodiments practical for basal body and a variety of other temperature measurements.

A dynamic temperature sensing control system such as system 300 of FIG. 3A advantageously provides a continuous update of the temperature values. However, such a system can be sensitive to latency and synchronicity of the audio output and input signals. As such, a dynamic temperature sensing control system or method that relies on response signal feedback can be adversely affected when implemented in electronic devices with poor, variable or unknown audio capabilities. In such cases a control system or method that works without the use of feedback can be desirable.

The temperature should preferably be assessed from the measurement of the bridge balance point, as otherwise gain factors and non-linearities in the audio signal channels will affect the readings. Without using any feedback, a control system may perform a sweep to determine the minimum balance point. This can be done with a variety of waveform amplitude shapes, constructed such that the beginning of the sweep can be determined without knowledge of the phase relationship between the input and the output signals. In some embodiments, such control systems are operable to measure temperature with a precision on the order of 0.1 degrees. An example of such a control system is discussed below with reference to FIGS. 3B-D.

FIG. 3B shows an example control system 300A for obtaining temperature measurements with thermometer circuit 305. Control system 300A comprises signal sources 340A and 340B which drive contacts of the audio interface (e.g. the left and right audio output ports) with harmonic driving signals D₁ and D₂ of a selected frequency, for example in the range from about 1000 Hz to about 5000 Hz. Driving signal D₁ is applied with substantially constant amplitude as shown in the left plot of FIG. 3C. The amplitude of driving signal D₁ may, for example, be about one half of a maximum amplitude that can be applied to the audio output contacts. Driving signal D₂ is initially applied with a substantially constant amplitude, then at time t₁ the amplitude of driving signal D₂ is adjusted to the maximum amplitude then ramped linearly downwardly to a minimum amplitude (e.g. 0) at time t₂, after which it returns to the substantially constant amplitude as shown in the right plot of FIG. 3C. The linear ramp shown in the right plot of FIG. 3C may be provided in driving signal D₂ periodically, and/or in response to a signal to initiate a temperature measurement.

A linear ramp is provided in the example amplitude profile shown in FIG. 3C, but other embodiments may provide different amplitude profiles. For example, in some embodiments the amplitude of one of the driving signals may sweep through less than the full range of amplitudes that can be applied to the audio output contacts. In some embodiments, instead of a linear ramp the amplitude may vary logarithmically, exponentially, or in another defined pattern.

The resulting response signal RES received at the microphone input is shown in FIG. 3D, and is characterized by a local minimum at time tₘᵢₙ with peaks on either side at times t₁ and t₂. A response measurement block 350A detects the initial spike in the response signal at time t₁ and a local minimum at time tₘᵢₙ. The response measurement block 350A determines the ratio of the amplitudes of the driving signals A(D₁)/A(D₂) based on the relative timings of the minimum at tₘᵢₙ and the initial spike at t₁ (or alternatively, based on the relative timings of the minimum at tₘᵢₙ and the trailing spike at t₂). The ratio of the amplitudes of the driving signals A(D₁)/A(D₂) is provided to a temperature calculation block 360 for determining the temperature as discussed above. Alternatively, the relative timings of the minimum and one or both of the spikes may be provided directly to the temperature calculation block.

The frequency of the driving signals in the FIG. 3C example are preferably about 1000-5000 Hz, and the linear ramp shown in the right plot of FIG. 3C preferably has a duration of about 0.05 to 0.5 seconds. Such driving signals allow multiple sets of data points to be obtained within one second. A stable temperature reading may thus be extracted by basic filtering such as, for example, moving average or median filtering, combinations thereof, finite impulse response (FIR) filtering, infinite impulse response (IIR) filtering, etc.

The temperature of a probe with a temperature sensitive element takes some time to reach equilibrium with the temperature of an area of the body it is in contact with. In some embodiments, predictive algorithms may be used to predict the temperatures, as disclosed for example in U.S. Pat. No. 7,318,004.

### Example Sphygmomanometry, Spirometry, and Perineometry Embodiments

A sphygmomanometer is a widely used device for the measurement of blood pressure, composed of an inflatable cuff to restrict blood flow, and a mercury or mechanical manometer to measure the pressure. Manual and semi-automatic devices are typically inflated with a bulb, while automated (digital) models typically use motorized inflation/deflation and read the pressure with an electronic sensor. Pressure measurements by sphygmomanometer may be used to determine a variety of physiological parameters relating to cardiovascular blood circulation including mean, systolic and diastolic blood pressure and heart rate. They may also be used to measure other physiological parameters such as respiration rate.

A spirometer is a widely used device used to measure various air flow characteristics of a patient's breathing. A typical spirometer functions as a flow meter that measures inspired and expired air volume over time. Spirometry may, for example, be used to measure a variety of respiratory parameters. Example measurements include:
TLC Total lung capacity: the volume of air in the lungs at maximal inflation
RV Residual volume: the volume of air remaining in the lungs after a maximal exhalation.
ERV Expiratory reserve volume: the maximal volume of air that can be exhaled from the end-expiratory position
IRV Inspiratory reserve volume: the maximal volume of air that can be inhaled from the end-inspiratory level
IC Inspiratory capacity: the sum of IRV and TV
IVC Inspiratory vital capacity: the maximum volume of air inhaled from the point of maximum expiration
VC Vital capacity: the volume equal to TLC-RV
VT Tidal volume: that volume of air moved into or out of the lungs during quiet breathing
FRC Functional residual capacity: the volume in the lungs at the end-expiratory position
RV/TLC % Residual volume expressed as percent of TLC
VA Alveolar gas volume
VL Actual volume of the lung including the volume of the conducting airway.
FVC Forced vital capacity: (e.g. from a maximally forced expiratory effort)
FEV1 Volume that has been exhaled at the end of the first second of forced expiration
FEFx Forced expiratory flow related to some portion of the FVC curve;
FEFmax The maximum instantaneous flow achieved during a FVC maneuver
FIF Forced inspiratory flow
PEF The highest forced expiratory flow measured with a peak flow meter
MVV Maximal voluntary ventilation: volume of air expired in a specified period during repetitive maximal effort.

A perineometer is a device for measuring voluntary contractions of the pelvic floor muscles. Perineometers may be used to measure the air pressure variations from an inflatable bladder, bulb or tube inserted into the vagina, or the mechanical strain variations from a strain gauge in a mechanically compressible member inserted into the vagina. Perineometric measurements may be used for such purposes as Kegel exercises or similar muscular function measurements. The sensor measurement data is then analyzed to determine the strength of the muscular contractions and other physiological parameters. In other embodiments, the strength of contractions of various other muscles may be measured by configuring air pressure or mechanical strain gauge sensors in a variety of devices that may be appropriately squeezed or compressed, including without limitation grips that measure forearm strength at the hand.

Example embodiments disclosed herein eliminate the need for conventional custom electronics of a digital sphygmomanometer, spirometer or perineometer by providing methods and systems for operating pressure sensors connectable to an electronic device such as a smartphone or media player by interfacing a pressure sensor directly through the audio interface of the device. Some embodiments use a commercially available pressure sensor comprising a piezoresistive bridge, which is operated in AC mode at standard audio signal levels, as described further below.

FIG. 4 shows an example pressure sensor 410 connected to two speaker contacts SPK, a microphone contact MIC and a ground contact of an electronic device 110. Pressure sensor 410 may, for example, comprise a piezoresistive bridge configured to measure any of gauge, differential and/or absolute pressure. Pressure sensor 410 may, for example comprise a commercially available piezoelectric pressure sensor (such as, for example, a MPX2010 Series Pressure Sensor from Freescale Semiconductor, Inc., or a 2SMPP MEMS Gauge Pressure Sensor from Omron Electronic Components LLC). Such pressure sensors are specified to be provided with DC supply voltage, but may be operated by providing them with harmonic driving signals D₁ and D₂ and measuring a response signal RES through the audio interface as described below. In some embodiments, systems used to process the signal from the pressure sensor comprise an AC auto-balancing bridge that continuously adjusts the amplitude and phase relationship of the two audio output channels in order to minimize the bridge response signal at the microphone input, and a dual-phase lockin-amplifier that does phase-locked detection of the response signal itself.

FIG. 4A shows an example control system 400 for obtaining blood pressure measurements with a piezoelectric pressure sensor 410A comprising a first pair of elements 412 and 414 connected to receive driving signal D₁ and a second pair of elements 416 and 418 connected received driving signal D₂. Control system 400 comprises signal sources 420A and 420B which drive contacts of the audio interface (e.g. the left and right audio output ports) with harmonic signals D₁ and D₂ of a selected frequency, typically in the range from about 100 Hz to about 10000 Hz.

A response cancellation block 430 receives a response signal from sensor 410A and controls signal sources 420A and/or 420B to minimize the response signal. The response signal is also multiplied by one of the driving signals (D₁ in the illustrated embodiment) and a phase shifted driving signal (generated by phase shift block 425) by multipliers 440A and 440B, respectively. The multiplied response signals are low pass filtered at filter blocks 450A and 450B, and then provided as a response input to a blood pressure extraction block 460. Blood pressure extraction block 460 is also provided with a balance input (e.g., the ratio of amplitudes of the driving signals) from response cancellation block 430.

At blood pressure extraction block 460, the balance input can be used to determine the absolute pressure at any given time. Lock-in amplification of the response signal may be used to provide a high resolution reading of the blood pressure modulation.

FIG. 4B is a graph showing exemplary output of blood pressure modulation which may be measured by a system such as system 400, with the solid trace (labeled ‘A’ in the legend of FIG. 4B) showing the in-phase component of an example lock-in amplified response signal, and the dashed trace (labeled ‘B’ in the legend of FIG. 4B) showing the out-of-phase component of the example lock-in amplified response signal. The systolic, mean and diastolic blood pressure as well has the heart rate can be extracted from this output using standard published techniques. Other physiological parameters such as respiration rate may also be extracted.

A system such as system 400 of FIG. 4A may also be connected to a differential pressure sensor for spirometry applications, with blood pressure extraction block 460 replaced or supplemented with an air flow and/or air volume calculation block configured to determine various respiratory parameters. Alternatively, a differential pressure sensor 410C may be coupled to a simplified control system 470 as shown in FIG. 4C for measurement of airflow. Control system 470 comprises a pair of signal sources 475A and 475B and a response cancellation block 480 which may be substantially similar to sources 340A and 340B and block 350 discussed above with respect to FIG. 3A, and as such will not be described again. Control system 470 also comprises a flow/volume calculation block 490 which is configured to determine various respiratory parameters from the ratio of driving amplitudes A(D₁)/A(D₂).

A system such as system 470 of FIG. 4C may also be connected to a pressure sensor coupled to an inflatable member or within a mechanically compressible member for perineometry applications, with flow/volume calculation block 490 replaced or supplemented with a calculation of the strength of voluntary muscle contractions in the patient based on the pressure sensor readings.

In an alternate embodiment, the perineometry sensor may comprise a strain gauge within a mechanically compressible member. The strain gauge sensor registers the degree of mechanical strain imposed by the muscular contractions. Strain gauges may be implemented in a variety of forms including mechanical, electrically resistive or capacitive, piezoresistive, fiber-optical, as well as other implementations of similar functionality. Strain gauge sensors may be implemented in an analogous manner as an air pressure sensor in a circuit similar to FIG. 4.

In example embodiments the stereo audio output is used to apply an AC voltage across the bridge of a sensor and the bridge response signal is recorded by the microphone input. No additional components are needed in these circuits. Methods and systems disclosed herein make it possible to replace the manometer of a conventional sphygmomanometer, or the sensor circuitry of a spirometer, with a small and inexpensive adaptor that plugs directly into the audio interface of a mobile or other electronic device.

FIG. 5 shows an example sphygmomanometry audio interface adaptor 500 according to one embodiment, and FIG. 6 shows an example spirometry audio interface adaptor 600 according to one embodiment. The adaptor 500 of FIG. 5 could also be used with a perineometer having an inflatable member, with or without the leak valve 550. Each of adaptors 500 and 600 comprise a standard TRRS-type audio plug 510 or 610 at one end. It is to be understood that different types of audio interface connectors may be used in other embodiments, as discussed above. Each of adaptors 500 and 600 also comprise a housing 520/620 containing a pressure sensor 530/differential pressure sensor 630. Pressure sensor 530 and differential pressure sensor 630 are directly wired to plugs 510 and 610, respectively, in the illustrated embodiments.

Adaptor 500 of FIG. 5 comprises a passage 540 in fluid communication with a pressure sensitive area of sensor 530. A tube connector 542 (e.g., a ¼ inch barbed hose adapter) may be provided to facilitate coupling a pressure tube connected to a cuff (not shown) to passage 540. Adaptor 500 may also comprise a leak valve 550 for allowing air to leave the pressure tube. While motorized pump inflation/deflation of the cuff is convenient, it consumes considerable power that would require a separate power source. For this reason a scheme of fixed bleed deflation has been implemented, wherein the cuff is manually inflated to a point higher than the systolic pressure of the subject, and a leak valve is then used to slowly ramp the pressure through the clinically relevant regime. Alternatively, a leak valve may be provided in the pressure tube. In other embodiments, the pressure sensor may be located outside the audio jack plug adaptor 500, as for example when the sensor may be connected to the jack plug or other adaptor via an electrical cable or other means.

Adaptor 600 of FIG. 6 comprises a pair of passages 640A and 640B in fluid communication with a pair of pressure sensitive areas of sensor 630. Tube connectors 642 (e.g., ¼ inch barbed hose adapters) may be provided to facilitate coupling pressure tubes 650A and 650B to passages 640A and 640B, respectively. Pressure tubes 650A and 650B are in fluid communication with a breathing tube 660 on opposite sides of a partial obstruction 670 as described above.

### Other Embodiments

The example embodiments discussed above are directed thermometry, sphygmomanometry, spirometry, and perineometry applications, but it is to be understood that other embodiments may be directed to other applications. For example, other types of sensors, including without limitation ECG sensors and EEG sensors, may be connected to audio interfaces of electronic devices in other embodiments.

FIG. 7 schematically illustrates an example electrocardiograph (ECG) sensor 700 connected to contacts of an electronic device 110 according to one embodiment. Sensor 700 comprises leads 701, 702, 703, and 704 (e.g. electrodes) which are coupled to a patient's body. Amplification circuits 710 and 712 are connected to leads 701-704 for measuring the differential voltage between the leads. In particular, circuit 710 generates a response signal 711 based on the differential voltage between leads 701 and 702, and circuit 712 generates a response signal 713 based on the differential voltage between leads 703 and 704. Response signals 711 and 713 are provided to transistors 716 and 718, respectively. Harmonic driving signals D₁ and D₂ generated by the audio output ports (for example, at a frequency in the range 100 Hz-100 kHz) are also respectively provided to transistors 716 and 718, where the driving signals D₁ and D₂ are modulated with the response signals 711 and 713. The resulting superposed modulated signals are received as a response signal at the microphone input, and the response signal is demodulated by a software-implemented control system to recover the original ECG waveforms. Such an arrangement overcomes the limiting audio system high-pass input filter, which will otherwise distort a low-frequency signal like an ECG waveform which may be typically 0.1 Hz to 50 Hz. Some systems in the prior art may utilize modulation of the original ECG signal to enable it to be carried through an analog system. However, they typically generate the modulation frequency within the sensor and do not drive the modulation circuit with the audio system. A similar implementation may be used for operating an EEG sensor to receive EEG signals through the audio system.

Although example embodiments have been described herein with the reference to the accompanying drawings, it is to be understood that the invention is not limited to those exact constructions and operations, and that various other changes and modifications may be made by one skilled in the art without departing from the scope or spirit of the invention.

Embodiments of the invention may be implemented using specifically designed hardware, configurable hardware, programmable data processors configured by the provision of software (which may optionally comprise ‘firmware’) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”) and the like. Examples of configurable hardware are: one or more programmable logic devices such as programmable array logic (“PALs”), programmable logic arrays (“PLAs”) and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

For example, while processes or blocks are presented in a given order, alternative examples may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Software and other modules may reside on servers, workstations, personal computers, tablet computers, data encoders, data decoders, PDAs, mobile phones, media players, and other devices suitable for the purposes described herein. Those skilled in the relevant art will appreciate that aspects of the system can be practiced with any suitable communications, data processing, or computer system configurations, including: Internet appliances, hand-held devices (including personal digital assistants (PDAs)), wearable computers, all manner of cellular or mobile phones, multi-processor systems, microprocessor-based or programmable consumer electronics (e.g., video projectors, audio-visual receivers, displays, such as televisions, and the like), set-top boxes, network PCs, mini-computers, mainframe computers, and the like.

Where a component (e.g. a software module, processor, controller, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

Specific examples of systems, methods and apparatus have been described herein for purposes of illustration. These are only examples. The technology provided herein can be applied to systems other than the example systems described above. Many alterations, modifications, additions, omissions and permutations are possible within the practice of this invention. This invention includes variations on described embodiments that would be apparent to the skilled addressee, including variations obtained by: replacing features, elements and/or steps with equivalent features, elements and/or steps; mixing and matching of features, elements and/or steps from different embodiments; combining features, elements and/or steps from embodiments as described herein with features, elements and/or steps of other technology; and/or omitting features, elements and/or steps from described embodiments.

It is therefore intended that the following appended claims and claims hereafter introduced are interpreted to include all such modifications, permutations, additions, omissions and sub-combinations as may reasonably be inferred. The scope of the claims should not be limited by the preferred embodiments set forth in the examples, but should be given the broadest interpretation consistent with the description as a whole. In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The above-described embodiments are intended to be examples only. Alterations, modifications and variations can be effected to the particular embodiments by those of skill in the art without departing from the scope, which is defined solely by the claims appended hereto.

What is claimed is:

1. A method for controlling an electronic device to operate an external sensor connectable to an audio interface of the electronic device, the audio interface comprising a plurality of contacts, the method comprising:
applying a first harmonic driving signal to a first contact and a second harmonic driving signal to a second contact of the audio interface for driving the external sensor;
receiving a response signal at a third contact of the audio interface;
adjusting at least one of the first and second harmonic driving signals;
determining one or more physiological parameters based on characteristics of the first and second harmonic driving signals and the response signal; and
outputting the determined one or more physical parameters.

2. A method according to claim 1 wherein adjusting comprises providing feedback based on the response signal for modifying one of the driving signals to minimize the response signal.

3. A method according to claim 1 wherein adjusting comprises maintaining a substantially constant amplitude of the first driving signal while sweeping an amplitude of the second harmonic driving signal through a predetermined range.

4. A method according to claim 3 wherein sweeping the amplitude comprises sweeping through a range between a maximum amplitude of the audio interface and zero, and the substantially constant amplitude is about one half of the maximum amplitude.

5. A method according to claim 3 wherein sweeping comprises linearly ramping the amplitude.

6. A method according to claim 3 comprising determining a ratio of driving signals based on timing information of a local minimum in the received response signal.

7. A method according to claim 3 wherein the external sensor comprises a temperature sensor, comprising determining a temperature based on the timing information of the local minimum in the received response signal.

8. A method according to claim 1 wherein the external sensor comprises a temperature sensor, comprising determining a temperature based on a ratio of amplitudes of the driving signals.

9. A method according to claim 7 wherein the temperature sensor consists essentially of a thermistor and a reference resistor.

10. A method according to claim 1 wherein the external sensor comprises a pressure sensor.

11. A method according to claim 10 comprising multiplying the response signal by one of the driving signals and a phase shifted version of one of the driving signals to generate a response input and determining one or more physiological parameters based on the response input.

12. A method according to claim 11 comprising determining mean blood pressure, systolic blood pressure, diastolic blood pressure, and/or heart rate based on the response input and a ratio of amplitudes of the driving signals.

13. A method according to claim 10 wherein the pressure sensor is coupled to receive pressure from an inflatable member.

14. A method according to claim 10 wherein the pressure sensor is within a mechanically compressible member.

15. A method according to claim 13 comprising determining a strength of muscular contractions based on a ratio of amplitudes of the driving signals.

16. A method according to claim 10 wherein the pressure sensor comprises a differential pressure sensor.

17. A method according to claim 11 wherein the pressure sensor comprises a differential pressure sensor, comprising determining respiratory parameters based on the response input and a ratio of amplitudes of the driving signals.

18. A system for controlling an electronic device to operate an external sensor connectable to an audio interface of the electronic device, the audio interface comprising a plurality of contacts, the system comprising:
a driving signal generator for applying a first harmonic driving signal to a first contact and a second harmonic driving signal to a second contact of the audio interface for driving the external sensor and adjusting at least one of the first and second harmonic driving signals;
a response signal detector for receiving a response signal at a third contact of the audio interface;
a physiological parameter extractor for determining one or more physiological parameters based on characteristics of the first and second harmonic driving signals and the response signal; and
an output for outputting the determined one or more physical parameters.

19. A system according to claim 18 wherein the driving signal generator receives feedback based on the response signal for modifying one of the driving signals to minimize the response signal.

20. A system according to claim 19 wherein the driving signal generator maintains a substantially constant amplitude of the first driving signal while sweeping an amplitude of the second harmonic driving signal through a predetermined range.

21. A system according to claim 20 wherein the driving signal generator sweeps the amplitude of the second harmonic driving signal through a range between a maximum amplitude of the audio interface and zero, and the substantially constant amplitude is about one half of the maximum amplitude.

22. A system according to claim 20 wherein the driving signal generator sweeps the amplitude of the second harmonic driving signal by linearly ramping the amplitude.

23. A system according to claim 20 wherein one of the response signal detector and the physiological parameter extractor determines a ratio of driving signals based on timing information of a local minimum in the received response signal.

24. A system according to claim 20 wherein the external sensor comprises a temperature sensor, wherein the physiological parameter extractor determines a temperature based on the timing information of the local minimum in the received response signal.

25. A system according to claim 18 wherein the external sensor comprises a temperature sensor, wherein the physiological parameter extractor determines a temperature based on a ratio of amplitudes of the driving signals.

26. A system according to claim 24 wherein the temperature sensor consists essentially of a thermistor and a reference resistor.

27. A system according to claim 18 wherein the external sensor comprises a pressure sensor.

28. A system according to claim 27 wherein the response signal detector multiplies the response signal by one of the driving signals and a phase shifted version of one of the driving signals to generate a response input and the physiological parameter extractor determines one or more physiological parameters based on the response input.

29. A system according to claim 28 wherein the physiological parameter extractor determines mean blood pressure, systolic blood pressure, diastolic blood pressure, and/or heart rate based on the response input and a ratio of amplitudes of the driving signals.

30. A system according to claim 27 wherein the pressure sensor is coupled to receive pressure from an inflatable member.

31. A system according to claim 27 wherein the pressure sensor is within a mechanically compressible member.

32. A system according to claim 30 wherein the physiological parameter extractor determines a strength of muscular contractions based on a ratio of amplitudes of the driving signals.

33. A system according to claim 27 wherein the pressure sensor comprises a differential pressure sensor.

34. A system according to claim 28 wherein the pressure sensor comprises a differential pressure sensor, wherein the physiological parameter extractor determines respiratory parameters based on the response input and a ratio of amplitudes of the driving signals.

35. A thermometer for connecting to an audio interface of an electronic device, the thermometer comprising:
a jack plug having first and second contacts for receiving first and second harmonic driving signals from the electronic device and a third contact for returning a response signal to the electronic device; and
a temperature sensor consisting essentially of a thermistor connected between the first and third contacts and a reference resistor connected between the second and third contacts.

36. An adaptor for connecting to an audio interface of an electronic device, the adaptor comprising:
a jack plug having first and second contacts for receiving first and second harmonic driving signals from the electronic device and a third contact for returning a response signal to the electronic device; and,
a pressure sensor having a pressure sensitive area, the pressure sensor connected as a bridge across the first and second contact to provide the response signal to the third contact, between the first and third contacts and a reference resistor connected between the second and third contacts.

37. An adaptor according to claim 36 comprising a housing encasing the pressure sensitive area and having a passage in fluid communication with the pressure sensitive area.

38. An adaptor according to claim 37 wherein the pressure sensor comprises a differential pressure sensor having two pressure sensitive areas, and wherein the housing has two passages, with one passage connected to each of the pressure sensitive areas.

39. An adaptor according to claim 37 wherein the housing comprises a tube connector for coupling the passage to a pressure tube.

40. An adaptor according to claim 36 comprising a mechanically compressible member, wherein the pressure sensor is contained within the mechanically compressible member.
